(19) United States
(12) Patent Application Publication
(10) Pub. No.: US 2003/0035795 A1
(43) Pub. Date: Feb. 20, 2003

(54) Methods for treating or reducing the risk of pain and inflammatory disorders by administering inhibitors of activated thrombin activatable fibrinolysis inhibitor

(76) Inventors: Stephen J. Gardell, Woodbridge, CT (US); Shi-Shan Mao, North Wales, PA (US)

Correspondence Address: MERCK AND CO INC, P O BOX 2000, RAHWAY, NJ, 070650907

(21) Appl. No.: 10/120,323

(22) Filed: Apr. 11, 2002

Related U.S. Application Data
(60) Provisional application No. 60/283,748, filed on Apr. 13, 2001.

(51) Int. Cl. A61K 38/55

(52) U.S. Cl. 424/94.1

(57) ABSTRACT

The invention includes methods for treating or reducing the risk of inflammation in a patient which comprises treating the patient with an inhibitor of activated thrombin activatable fibrinolysis inhibitor. Such diseases include but are not limited to nephritis, systemic lupus erythematosus, rheumatoid arthritis, glomerulonephritis, and sacoidosis. The invention includes methods for treating or reducing the risk of pain in a patient which comprises treating the patient with an inhibitor of activated thrombin activatable fibrinolysis inhibitor. In one class of these methods, the inhibitor of activated thrombin activatable fibrinolysis inhibitor is selected from the group consisting of 2-(6-amino-pyridin-3-ylmethyl)-3-butyl-succinic acid, 2-(6-amino-pyridin-3-ylmethyl)-3-phenethyl-succinic acid, 2-(6-amino-pyridin-3-ylmethyl)-3-methyl-succinic acid, 2-(6-amino-5-methyl-pyridin-3-ylmethyl)-3-[(1-benzyloxycarbonylamino-2-methyl-propyl)hydroxy-phosphinoyl]-propionic acid, 2-(6-amino-pyridin-3-ylmethyl)-3-[hydroxy-(3-phenyl-propyl)-phosphinoyl]-propionic acid, 2-(amino-pyridin-3-ylmethyl)-N-hydroxy-succinamic acid, 3-(6-amino-pyridin-3-yl)-2-mercaptomethyl-propionic acid, 2-(2-amino-pyridin-4-ylmethyl)-3-mercapto-propionic acid, 2-(6-amino-pyridin-3-ylmethyl)-2-mercaptomethyl-butyric acid, 3-(6-amino-5-methyl-pyridin-3-yl)-2-mercaptomethyl-2-methyl-propionic acid, 3-(6-amino-5-methyl-pyridin-3-yl)-2-mercaptomethyl-propionic acid, 3-(6-amino-4-methyl-pyridin-3-yl)-2-mercaptomethyl-propionic acid, and 3-(6-amino-pyridin-3-yl)-2-mercaptomethyl-butyric acid or a pharmaceutically acceptable salt thereof.

The invention is also a method for treating or reducing the risk of inflammation in a patient, or treating or reducing the risk of pain, which comprises treating the patient with a composition comprising an inhibitor of activated thrombin activatable fibrinolysis inhibitor and an NSAID, e.g., a COX-2 inhibitor. Such diseases include but are not limited to nephritis, systemic lupus, erythematosus, rheumatoid arthritis, glomerulonephritis, and sacoidosis.

### BACKGROUND OF THE INVENTION

This invention relates to methods for treating and reducing the risk of pain and inflammatory disorders by administering an inhibitor of activated thrombin activatable fibrinolysis inhibitor.

Anti-inflammatory drugs include non steroidal anti-inflammatory drugs (NSAIDs) which exert anti-inflammatory, analgesic and antipyretic activity. These include salicylates such as aspirin, sodium salicylate, choline salicylate, salicylsalicylic acid, diflunisal, and salsalate; indoleacetic acids such as indomethacin and sulindac; pyrazoles such as phenylbutazone, oxyphenbutazone; pyrrolealkanoic acids such as tolmetin; phenylacetic acids such as ibuprofen, feroprofen, flurbiprofen, and ketoprofen; fenamates such as mefanamic acid, and meclofenamate; oxicams such as piroxicam; and naphthaleneacetic acids such as naproxen. Nearly all act by inhibiting cyclo-oxygenase activity. Aspirin, for example, acetylates and irreversibly inactivates cyclo-oxygenase. Others, such as indomethacin, inhibit cyclo-oxygenase activity reversibly by binding in a stereospecific manner to one or another subunit of the enzyme. NSAIDs are active in reducing the prostaglandin-induced pain and swelling associated with the inflammation process but are also active in affecting other prostaglandin-regulated processes not associated with the inflammation process. Thus, use of high doses of most common NSAIDs can produce severe side effects, including life threatening ulcers, that limit their therapeutic potential.

Adrenal corticosteroids, which are alternatives to NSAIDs for treating inflammatory diseases, have even more drastic side effects, especially when long term therapy is involved. These steroids, including hydrocortisone, prednisolone, 6 alpha-methylprednisolone, triamcinolone, dexamethasone and betaroethasone, affect inflammation by a possible mechanism whereby they bind to intracellular glucocorticoid receptors to subsequently initiate a series of cellular events involving synthesis of new phospholipid inhibitory proteins, or lipocortins, that can affect the inflammatory and the teratogenic responses of certain cells exposed to glucocorticoids. The anti-inflammatory effect of glucocorticoids has been well documented.

Excessive bleeding disorders are associated with development of inflammatory conditions. Hemophilia, a bleeding disorder caused by a deficiency clotting Factor VIII or clotting Factor IX, can result in recurring bleeding into joints and muscles that causes crippling inflammation and deformities. Hemophilia also causes swelling of the base of the tongue until it blocks the airway.

Fibrinolysis is the result of a series of enzymatic reactions resulting in the degradation of fibrin by plasmin. The activation of plasminogen is the central process in fibrinolysis. The cleavage of plasminogen to produce plasmin is accomplished by the plasminogen activators, tissue-type plasminogen activator (t-PA) or urokinase-type plasminogen activator (u-PA). Initial plasmin degradation of fibrin generates carboxy-terminal lysine residues that serves as high affinity binding sites for plasminogen. Since plasminogen bound to finbrin is much more readily activated to plasmin than free plasminogen this mechanism provides a positive feedback regulation of fibrinolysis.

One of the endogenous inhibitors to fibrinolysis is carboxypeptidase U (CPU). CPU is also known as active thrombin activatable fibrinolysis inhibitor (TAFIa). CPU is formed during coagulation and fibrinolysis from its precursor proCPU by the action of proteolytic enzymes, e.g., thrombin, thrombin-thrombomodulin complex or plasmin. CPU cleaves basic amino acids at the carboxy-terminal of fibrin fragments. The loss of carboxy-terminal lysines and thereby of lysine binding sites for plasminogen then serves to inhibit fibrinolysis. By inhibiting the loss of lysine binding sites for plasminogen and thus increase the rate of plasmin formation, effective inhibitors of carboxypeptidase U would be expected to facilitate fibrinolysis.

The principal pharmacological effects of nonsteroidal anti-inflammatory drugs are due to their ability to inhibit prostaglandin synthesis by blocking cyclooxygenase. Such compounds exhibit anti-inflammatory, analgesic, and antipyretic activities in animal models. Compounds which selectively inhibit cyclooxygenase-2 over cyclooxygenase-1 are particularly suited for treating inflammation and related conditions since they do not inhibit cyclooxygenase-1, thereby avoiding undesirable gastrointestinal side effects associated with cyclooxygenase-1 inhibition.

As described below, it has now been found that inhibitors of activated thrombin activatable fibrinolysis inhibitor, which inhibit thrombosis, are effective for treating and preventing pain and inflammation.

### SUMMARY OF THE INVENTION

The invention is a method for treating an inflammatory disease in a patient which comprises treating the patient with an inhibitor of activated thrombin activatable fibrinolysis inhibitor. Such diseases include but are not limited to nephritis, systemic lupus erythematosus, rheumatoid arthritis, glomerulonephritis, and sacoidosis.

The invention is also a method for preventing inflammation, precluding symptoms of inflammation, reducing the severity of symptoms, reducing the risk of inflammation, relieving inflammation, preventing pain, precluding pain, reducing the severity of pain, reducing the risk of pain, or relieving pain in a patient which comprises treating the patient with an inhibitor of activated thrombin activatable fibrinolysis inhibitor.

The invention is also a method for reducing the risk of an inflammatory disease in a patient which comprises treating the patient with an inhibitor of activated thrombin activatable fibrinolysis inhibitor.

The invention is also a method for treating pain in a patient which comprises treating the patient with an inhibitor of activated thrombin activatable fibrinolysis inhibitor.

The invention is also a method for reducing the risk of pain in a patient which comprises treating the patient with an inhibitor of activated thrombin activatable fibrinolysis inhibitor.

In one class of the method, the inhibitor of activated thrombin activatable fibrinolysis inhibitor is selected from the group consisting of 2-(6-amino-pyridin-3-ylmethyl)-3-butyl-succinic acid, 2-(6-amino-pyridin-3-ylmethyl)-3-phenethyl-succinic acid, 2-(6-amino-pyridin-3-ylmethyl)-3-methyl-succinic acid, 2-(6-amino-5-methyl-pyridin-3-ylmethyl)-3-[(1-benzyloxycarbonylamino-2-methyl-propyl)hydroxy-phosphinoyl]-propionic acid, 2-(6-amino-pyridin-3-ylmethyl)-3-[hydroxy-(3-phenyl-propyl)-phosphinoyl]-propionic acid, 2-(amino-pyridin-3-ylmethyl)-N-hydroxy-succinamic acid, 3-(6-amino-pyridin-3-yl)-2-mercaptomethyl-propionic acid, 2-(2-amino-pyridin-4-ylmethyl)-3-mercapto-propionic acid, 2-(6-amino-pyridin-3-ylmethyl)-2-mercaptomethyl-butyric acid, 3-(6-amino-5-methyl-pyridin-3-yl)-2-mercaptomethyl-2-methyl-propionic acid, 3-(6-amino-5-methyl-pyridin-3-yl)-2-mercaptomethyl-propionic acid, 3-(6-amino-4-methyl-pyridin-3-yl)-2-normally expressed in basal conditions but are induced at sites of tissue injury and inflammation under the influence of agents such as cytokines and lipopolysaccharide. Proteases that are known to activate thrombin activatable fibrinolysis inhibitor (thrombin and plasmin) are generated at sites of tissue injury. Thus, inhibitors of activated thrombin activatable fibrinolysis inhibitor are effective for treating pain, fever and inflammation of a variety of conditions.

The present invention is a method for relieving pain, fever and inflammation of a variety of conditions including nephritis, systemic lupus erythematosus, rheumatoid arthritis, glomerulonephritis, sacoidosis, rheumatic fever, symptoms associated with influenza or other viral infections, common cold, low back and neck pain, dysmenorrhea, headache, toothache, sprains and strains, myositis, neuralgia, synovitis, arthritis, including rheumatoid arthritis degenerative joint diseases (osteoarthritis), gout and ankylosing spondylitis, bursitis, burns, injuries, following surgical and dental procedures in a patient, e.g., a human, by administering to the patient a therapeutically effective amount of an inhibitor of activated thrombin activatable fibrinolysis inhibitor. Inhibitors of activated thrombin activatable fibrinolysis inhibitor may also be useful for the treatment of dementia including pre-senile and senile dementia, and in particular, dementia associated with Alzheimer Disease.

The present invention is also a method for relieving pain, fever and inflammation of a variety of conditions including nephritis, systemic lupus erythematosus, rheumatoid arthritis, glomerulonephritis, sacoidosis, rheumatic fever, symptoms associated with influenza or other viral infections, common cold, low back and neck pain, dysmenorrhea, headache, toothache, sprains and strains, myositis, neuralgia, synovitis, arthritis, including rheumatoid arthritis degenerative joint diseases (osteoarthritis), gout and ankylosing spondylitis, bursitis, burns, injuries, following surgical and dental procedures in a patient, e.g., a human, having one or more of these conditions in association with a second condition in which provision of an antithrombotic effect is also desirable. The present invention is also a method for relieving pain, fever and inflammation of a variety of such conditions, in a patient, e.g., a human, having one or more of these conditions in association with a second condition in which provision of an antithrombotic effect to prevent or reduce the incidence of thrombosis is also desirable.

In inflammatory diseases wherein fibrin formation is prominent, the fibrin may be a determinant of the pathology. Fibrin serves as a matrix onto which inflammatory cells can migrate and adhere. (see Sherman et al., 1977 J. Exp. Med. 145:76-85; Altieri et al., 1986 J. Clin. Invest. 78:968-976; Wright et al., 1983 Proc. Natl. Acad. Sci. 85:7734-7738; Altieri et al., 1993 J. Biol. Chem. 268;1847-1853). Fibrin also enhances expression of the inflammatory cytokine IL-1beta and decreases expression of IL-1 receptor antagonist by human peripheral blood mononuclear cells (see Perez 1995 J. immunol. 154:1879-1887). The anticoagulants warfarin and heparin attenuate delayed-type hypersensitivity reactions and experimental nephritis in animals. (see Jasain et al., Immunopathogenesis of Rheumatoid Arthritis Eds. G. S. Panayi et al., Surrey, U K, Reedbooks, Ltd. and Halpern et al., 1965 Nature 205:257-259). Enzymatic defibrination with ancrod diminishes the degree of experimental nephritis (Naish et al., 1972 Clin. Sci. 42:643-646), systemic lupus erythematosus (Cole et al., 1990 Kidney Int. 37:29-35, and rheumatoid arthritis (see Busso et al., 1998 J. Clin. Invest. 102:41-50) in animals, and glomerulonephritis in man (see Kim et al., 1988 Q. J. Med. 69:879-905). Additionally, intra articular injection of fibrin induces arthritis in rabbits immunized with fibrin Dumonde et al., 1961 British Journal of Experimental Pathology XLIII:373-383), and antigen-induced arthritis in mice is exacerbated in urokinase-deficient mice wherein fibrinolysis synovial fibrin is compromised (see Busso et al., 1998 J. Clin. Invest. 102:41-50).

In diseases where fibrin deposition is prominent such as, but not limited to, rheumatoid arthritis, systemic lupus erythematosus, glomerulonephritis, vasculitis and sacoidosis, lowering the steady state concentration of fibrin by administration of an inhibitor of activated thrombin activatable fibrinolysis inhibitor will, according to the instant invention, diminish the pathological inflammatory responses associated with these diseases.

Inhibitors of Activated Thrombin Activatable Fibrinolysis Inhibitor

Inhibitors of activated thrombin activatable fibrinolysis inhibitor include those showing an inhibiting effect according to the assay described in Hendriks et al., Clinical Chemistry, 31, 1936-1939(1985) and Wang et al., The Journal of Biological Chemistry, 269, 15937-15944 (1994). The contents of these two publications are hereby incorporated by reference.

Inhibitors of activated thrombin activatable fibrinolysis inhibitor include those, for example, described in WO 00/66550 (e.g., 2-(6-amino-pyridin-3-ylmethyl)-3-butyl-succinic acid, 2-(6-amino-pyridin-3-ylmethyl)-3-phenethyl-succinic acid, 2-(6-amino-pyridin-3-ylmethyl)-3-methyl-succinic acid, 2-(6-amino-5-methyl-pyridin-3-ylmethyl)-3-[(1-benzyloxycarbonylamino-2-methylpropyl)hydroxy-phosphinoyl]-propionic acid, 2-(6-amino-pyridin-3-ylmethyl)-3-[hydroxy-(3-phenyl-propyl)-phosphinoyl]-propionic acid, 2-(amino-pyridin-3-ylmethyl)-N-hydroxy-succinamic acid) and WO 00/66557 (e.g., 3-(6-amino-pyridin-3-yl)-2-mercaptomethyl-propionic acid, 2-(2-amino-pyridin-4-ylmethyl)-3-mercapto-propionic acid, 2-(6-amino-pyridin-3-ylmethyl)-2-mercaptomethyl-butyric acid, 3-(6-amino-5-methyl-pyridin-3-yl)-2-mercaptomethyl-2-methyl-propionic acid, 3-(6-amino-5-methyl-pyridin-3-yl)-2-mercaptomethyl-propionic acid, 3-(6-amino-4-methyl-pyridin-3-yl)-2-mercaptomethyl-propionic acid, and 3-(6-amino-pyridin-3-yl)-2-mercaptomethyl-butyric acid), both of which define inhibitors of activated thrombin activatable fibrinolysis inhibitor to include compounds having the formula C(XR¹)(R²)(R³)(YR⁴). The contents of WO 00/66550 and WO 00/66557 are also hereby incorporated by reference.

These inhibitors are therapeutically useful in treating those conditions where inhibition of activated thrombin activatable fibrinolysis inhibitor is beneficial, such as in the treatment of prophylaxis of thrombosis and hypercoagulability in blood and tissues of mammals, including man.

It is known that hypercoagulability may lead to thrombo-embolic diseases. Conditions associated with hypercoagulability and thrombo-embolic diseases which may be mentioned include protein C resistance and inherited or acquired deficiencies in antithrombin III, protein C, protein S and heparin cofactor II. Other conditions known to be associated with hyper-coagulability and thrombo-embolic disease include circulatory and septic shock, circulating antiphospholipid antibodies, homocysteinami, heparin induced thrombocytopenia and defects in fibrinolysis. The inhibitors are thus indicated both in the therapeutic and/or prophylactic treatment of these conditions. The inhibitors are further indicated in the treatment of conditions where there is an undesirable excess of proCPU/CPU.

Particular disease states which may be mentioned include the therapeutic and/or prophylactic treatment of venous thrombosis and pulmonary embolism, arterial thrombosis (e.g., in myocardial infarction, unstable angina, thrombosis-based stroke ad peripheral arterial thrombosis) and systemic embolism usually from the atrium during arterial fibrillation or from the left ventricle after transmural myocardial infarction.

Moreover, the inhibitors are useful in prophylaxis of re-occlusion and restenosis (i.e., thrombosis) after thrombolysis, percutaneous trans-luminal angioplasty (PTA) and coronary bypass operations; the prevention of re-thrombosis after microsurgery and vascular surgery in general.

Further indications include the therapeutic and/or prophylactic treatment of disseminated intravascular coagulation caused by bacteria, multiple trauma, intoxication or any other mechanism, fibrinolytic treatment when blood is in contact with foreign surfaces in the body, such as vascular grafts, vascular stents, vascular catheters, mechanical and biological prosthetic valves or any other medical device, and fibrinolytic treatment when blood is in contact with medical devices outside the body, such as during cardiovascular surgery using a heart-lung machine or in haemodialysis.

The inhibitors of activated thrombin activatable fibrinolysis inhibitor of the invention can be administered in such oral forms as tablets, capsules (each of which includes sustained release or timed release formulations), pills, powders, granules, elixers, tinctures, suspensions, syrups, and emulsions. Likewise, they may be administered in intravenous (bolus or infusion), intraperitoneal, subcutaneous, or intramuscular form, all using forms well known to those of ordinary skill in the pharmaceutical arts. An effective but non-toxic amount of the compound desired can be employed as an anti-aggregation agent. For treating ocular build up of fibrin, the compounds may be administered intraocularly or topically as well as orally or parenterally.

The inhibitors of activated thrombin activatable fibrinolysis inhibitor can be administered in the form of a depot injection or implant preparation which may be formulated in such a manner as to permit a sustained release of the active ingredient. The active ingredient can be compressed into pellets or small cylinders and implanted subcutaneously or intramuscularly as depot injections or implants. Implants may employ inert materials such as biodegradable polymers or synthetic silicones, for example, Silastic, silicone rubber or other polymers manufactured by the Dow-Coming Corporation.

The inhibitors of activated thrombin activatable fibrinolysis inhibitor can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The inhibitors of activated thrombin activatable fibrinolysis inhibitor may also be delivered by the use of monoclonal antibodies as individual carriers to which the compound molecules are coupled. The inhibitors of activated thrombin activatable fibrinolysis inhibitor may also be coupled with soluble polymers as targetable drug carriers. Such polymers can include polyinlypyrrolidone, pyran copolymer, polyhydroxy-propyl-methacrylamide-phenol, polyhydroxyethyl-aspartamide-phenol, or polyethyleneoxide-polylysine substituted with palmitoyl residues. Furthermore, the inhibitors of activated thrombin activatable fibrinolysis inhibitor may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels.

The dosage regimen utilizing the inhibitors of activated thrombin activatable fibrinolysis inhibitor is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Oral dosages of the inhibitors of activated thrombin activatable fibrinolysis inhibitor, when used for the indicated effects, will range between about 0.01 mg per kg of body weight per day (mg/kg/day) to about 30 mg/kg/day, preferably 0.025-7.5 mg/kg/day, more preferably 0.1-2.5 mg/kg/day, and most preferably 0.1-1.0 mg/kg/day (unless specified otherwise, amounts of active ingredients are on free base basis). For example, an 80 kg patient would receive between about 0.8 mg/day and 2.4 g/day, preferably 2-600 mg/day, more preferably 8-200 mg/day, and most preferably 8-80 mg/day. A suitably prepared medicament for once a day administration would thus contain between 0.8 mg and 2.4 g, preferably between 2 mg and 600 mg, more preferably between 8 mg and 200 mg, and most preferably 8 mg and 80 mg, e.g., 8 mg, 20 mg, 40 mg and 80 mg. Advantageously, the inhibitors of activated thrombin activatable fibrinolysis inhibitor may be administered in divided doses of two, three, or four times daily. For administration twice a day, a suitably prepared medicament would contain between 0.4 mg and 4 g, preferably between 1 mg and 300 mg, more preferably between 4 mg and 100 mg, and most preferably 4 mg and 40 mg, e.g., 4 mg, 10 mg, 20 mg and 40 mg.

Intravenously or subcutaneously, the patient would receive the active ingredient in quantities sufficient to deliver between 0.025-7.5 mg/kg/day, preferably 0.1-2.5 mg/kg/day, and more preferably 0.1-1.0 mg/kg/day. Such quantities may be administered in a number of suitable ways, e.g. large volumes of low concentrations of active ingredient during one extended period of time or several times a day, low volumes of high concentrations of active ingredient during a short period of time, e.g. once a day. Typically, a conventional intravenous formulation may be prepared which contains a concentration of active ingredient of between about 0.01-1.0 mg/ml, e.g. 0.1 mg/ml, 0.3 mg/ml, and 0.6 mg/ml, and administered in amounts per day of between 0.01 ml/kg patient weight and 10.0 ml/kg patient weight, e.g. 0.1 ml/kg, 0.2 ml/kg, 0.5 ml/kg. In one example, an 80 kg patient, receiving 8 ml twice a day of an intravenous formulation having a concentration of active ingredient of 0.5 mg/ml, receives 8 mg of active ingredient per day. Glucuronic acid, L-lactic acid, acetic acid, citric acid or any pharmaceutically acceptable acid/conjugate base with reasonable buffering capacity in the pH range acceptable for intravenous administration may be used as buffers. Consideration should be given to the solubility and chemical compatibility of the drug in choosing an appropriate excipient. Subcutaneous formulations, preferably prepared according to procedures well known in the art at a pH in the range between 7.0 and 7.4, also include suitable buffers and isotonicity agents. They are formulated to deliver a daily dose of inhibitors of activated thrombin activatable fibrinolysis inhibitor in one or more daily subcutaneous administrations, e.g., one, two or three time each day. The choice of appropriate buffer and pH of a formulation, depending on solubility of the drug to be administered, is readily made by a person having ordinary skill in the art.

The compounds can also be administered in intranasal form via topical use of suitable intranasal vehicles, or via transdermal routes, using those forms of transdermal skin patches well known to those of ordinary skill in that art. To be administered in the form of a transdermal delivery system, the dosage administration will, or course, be continuous rather than intermittent throughout the dosage regime.

The inhibitors of activated thrombin activatable fibrinolysis inhibitor are typically administered as active ingredients in admixture with suitable pharmaceutical diluents, excipients or carriers (collectively referred to herein as “carrier” materials) suitably selected with respect to the intended form of administration, that is, oral tablets, capsules, elixers, syrups and the like, and consistent with convention pharmaceutical practices.

For instance, for oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, gelatin, natural sugars such as glucose or beta-lactose, corn-sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch methyl cellulose, agar, bentonite, xanthan gum and the like.

Typical uncoated tablet cores suitable for administration of inhibitors of activated thrombin activatable fibrinolysis inhibitor are comprised of, but not limited to, the following range amounts of standard ingredients:

| Excipient | general | preferred | most preferred |
|---|---|---|---|
| mannitol | 10-90% | 25-75% | 30-60% |
| microcrystalline | 10-90 | 25-75 | 30-60 |
| cellulose | | | |
| magnesium stearate | 0.1-5.0 | 0.1-2.5 | 0.5-1.5 |

Mannitol, microcrystalline cellulose and magnesium stearate may be substituted with alternative pharmaceutically acceptable excipients.

All of the inhibitors of activated thrombin activatable fibrinolysis inhibitor, cellulose, and a portion of the corn starch are mixed and granulated to 10% corn starch paste. The resulting granulation is sieved, dried and blended with the remainder of the corn starch and the magnesium stearate. The resulting granulation is then compressed into tablets containing 25.0, 50.0, and 100.0 mg, respectively, of active ingredient per tablet.

Phosphate buffers and various other buffer acids, such as L-lactic acid, acetic acid, glucuronic acid or any pharmaceutically acceptable acid/conjugate base with reasonable buffering capacity in the pH range acceptable for subcutaneous administration may be substituted for citric acid.

“Pharmaceutically acceptable salts” means non-toxic salts of the compounds employed in this invention which are generally prepared by reacting the free acid with a suitable organic or inorganic base. Examples of salt forms of inhibitors of activated thrombin activatable fibrinolysis inhibitor may include, but are not limited to, acetate, benzenesulfonate, benzoate, bicarbonate, bisulfate, bitartrate, borate, bromide, calcium, calcium edetate, camsylate, carbonate, chloride, clavulanate, citrate, dihydrochloride, edetate, edisylate, estolate, esylate, fumarate, gluceptate, gluconate, glutamate, hexylresorcinate, hydrabamine, hydrobromide, hydrochloride, hydroxynapthoate, isothionate, lactate, lactobionate, laurate, malate, maleate, mandelate, mesylate, methylsulfate, mucate, oleate, oxalate, pamaote, palmitate, panthothenate, phosphate/diphosphate, polygalacturonate, potassium, salicylate, sodium, stearate, subacetate, succinate, tannate, tartrate, teoclate, tosylate, and valerate. Examples of salt forms of COX-2 inhibitors include but are not limited to salts derived from inorganic bases including aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

Unless defined otherwise, “therapeutically effective amount” means that amount of a drug or pharmaceutical agent that will elicit the biological or medical response of a tissue, a system, animal or human that is being sought by a researcher, veterinarian, medical doctor or other clinician.

Unless defined otherwise, “prophylactically effective amount” means that amount of a pharmaceutical drug that will prevent or reduce the risk of occurrence of the biological or medical event that is sought to be prevented in a tissue, a system, animal or human by a researcher, veterinarian, medical doctor or other clinician.

The term “patient” means a subject, including a human, who is experiencing an inflammatory condition or pain, such as inflammation or pain resulting from conditions including but not limited to nephritis, systemic lupus erythematosus, rheumatoid arthritis, glomerulonephritis, sacoidosis, rheumatic fever, symptoms associated with influenza or other viral infections, common cold, low back and neck pain, dysmenorrhea, headache, toothache, sprains and strains, myositis, neuralgia, synovitis, arthritis, including rheumatoid arthritis degenerative joint diseases (osteoarthritis), gout and ankylosing spondylitis, bursitis, burns, injuries, or surgical and dental procedures, or a subject, including a human, who is predisposed to experience an inflammatory condition or pain, such as inflammation or pain resulting from conditions including but not limited to nephritis, systemic lupus erythematosus, rheumatoid arthritis, glomerulonephritis, sacoidosis, rheumatic fever, symptoms associated with influenza or other viral infections, common cold, low back and neck pain, dysmenorrhea, headache, toothache, sprains and strains, myositis, neuralgia, synovitis, arthritis, including rheumatoid arthritis degenerative joint diseases (osteoarthritis), gout and ankylosing spondylitis, bursitis, burns, injuries, or surgical and dental procedures.

The term “preventing inflammation” means precluding an inflammatory condition, or reducing the severity of the condition, associated with an inflammatory disease.

The term “precluding symptoms of inflammation” means making the experience of symptoms of inflammation impossible or largely ineffectual by removing the conditions needed for them.

The term “reducing the severity of symptoms” means substantially lowering the degree of inflammation symptoms associated with an inflammatory disease.

The term “reducing the risk of inflammation” means substantially lowering the tendency of patients susceptible to an inflammatory condition, to experience inflammation.

The term “relieving inflammation” means eliminating or substantially eliminating inflammation.

The term “preventing pain” means precluding pain, or reducing the severity of pain, associated with an inflammatory disease.

The term “precluding pain” means making the experience of pain impossible or largely ineffectual by removing the conditions needed for them.

The term “reducing the severity of pain” means substantially lowering the degree of pain associated with an inflammatory disease.

The term “reducing the risk of pain” means substantially lowering the tendency of patients susceptible to an inflammatory condition, to experience pain.

The term “relieving pain” means eliminating or substantially eliminating pain.

Similarly, inhibitors of activated thrombin activatable fibrinolysis inhibitor will be useful as a partial or complete substitute for conventional NSAIDs in preparations wherein they are presently co-administered with other agents or ingredients. Thus in further aspects, the invention encompasses pharmaceutical compositions for treating inflammatory diseases as defined above comprising a non-toxic therapeutically effective amount of an inhibitor of activated thrombin activatable fibrinolysis inhibitor as defined above and one or more ingredients such as another pain reliever including acetominophen or phenacetin; a potentiator including caffeine; an H2-antagonist, aluminum or magnesium hydroxide, simethicone, a decongestant including phenylephrine, phenylpropanolamine, pseudophedrine, oxymetazoline, ephinephrine, naphazoline, xylometazoline, propylhexedrine, or levo-desoxyephedrine; an antiitussive including codeine, hydrocodone, caramiphen, carbetapentane, or dextramethorphan; a diuretic; a sedating or non-sedating antihistamine. In addition the invention encompasses a method of treating inflammatory diseases comprising administration to a patient in need of such treatment a non-toxic therapeutically effect amount of an inhibitor of activated thrombin activatable fibrinolysis inhibitor, optionally co-administered with one or more of such ingredients as listed immediately above.

The instant invention also involves a novel combination therapy comprising the administration of a therapeutically effective amount of an NSAID such as a COX-2 inhibitor in combination with a therapeutically effective amount of an inhibitor of activated thrombin activatable fibrinolysis inhibitor to a mammal, and more particularly, to a human. The combination therapy is used to treat inflammatory diseases.

Combination

The instant pharmaceutical combinations comprising an inhibitor of activated thrombin activatable fibrinolysis inhibitor in combination with an NSAID such as a COX-2 inhibitor include administration of a single pharmaceutical dosage formulation which contains both the inhibitor of activated thrombin activatable fibrinolysis inhibitor and the NSAID, as well as administration of each active agent in its own separate pharmaceutical dosage formulation. Where separate dosage formulations are used, the inhibitor of activated thrombin activatable fibrinolysis inhibitor and the NSAID can be administered at essentially the same time, i.e., concurrently, or at separately staggered times, i.e, sequentially. The “instant pharmaceutical combination” is understood to include all these regimens. Administration in these various ways are suitable for the present invention as long as the beneficial pharmaceutical effect of the inhibitor of activated thrombin activatable fibrinolysis inhibitor and the NSAID are realized by the patient at substantially the same time. Such beneficial effect is preferably achieved when the target blood level concentrations of each active drug are maintained at substantially the same time. It is preferred that the inhibitor of activated thrombin activatable fibrinolysis inhibitor and the NSAID be co-administered concurrently on a once-a-day dosing schedule; however, varying dosing schedules, such as the inhibitor of activated thrombin activatable fibrinolysis inhibitor once per day and the NSAID once, twice or more times per day, or the NSAID once per day and the inhibitor of activated thrombin activatable fibrinolysis inhibitor once, twice or more times per day, is also encompassed herein. A single oral dosage formulation comprised of both the inhibitor of activated thrombin activatable fibrinolysis inhibitor and the NSAID is preferred. A single dosage formulation will provide convenience for the patient.

The instant invention also provides pharmaceutical compositions comprised of a therapeutically effective amount of an NSA/D, or a pharmaceutically acceptable salt thereof, in combination with a therapeutically effective amount of an inhibitor of activated thrombin activatable fibrinolysis inhibitor, or a pharmaceutically acceptable salt thereof, and a pharmaceutically acceptable carrier. One embodiment of the instant compositions is a single composition adapted for oral administration comprised of a therapeutically effective amount of a COX-2 inhibitor in combination with a therapeutically effective amount of a inhibitor of activated thrombin activatable fibrinolysis inhibitor and a pharmaceutically acceptable carrier. The combination can also be administered in separate dosage forms, each having one of the active agents. If administered in separate dosage forms, the separate dosage forms are administered such that the beneficial effect of each active agent is realized by the patient at substantially the same time.

NSAIDs

Common NSAIDs include salicylates such as aspirin, sodium salicylate, choline salicylate, salicylsalicylic acid, diflunisal, and salsalate; indoleacetic acids such as indomethacin and sulindac; pyrazoles such as phenylbutazone, oxyphenbutazone; pyrrolealkanoic acids such as tolmetin; phenylacetic acids such as ibuprofen, feroprofen, flurbiprofen, and ketoprofen; fenamates such as mefanamic acid, and meclofenamate; oxicams such as piroxicam; and naphthaleneacetic acids such as naproxen. NSAIDs are characterized by their ability to inhibit prostaglandin synthesis by inhibiting cyclooxygenase activity.

COX-2 Inhibitors

Employing the human whole blood COX-1 assay and the human whole blood COX-2 assay described in C. Brideau et al, Inflamm. Res. 45: 68-74 (1996), herein incorporated by reference, preferably, the compounds have a cyclooxygenase-2 IC50 of less than about 21M in the human whole blood COX-2 assay, yet have a cyclooxygenase-1 IC50 of greater than about 5 μM in the human whole blood COX-1 assay. Also preferably, the compounds have a selectivity ratio of cyclooxygenase-2 inhibition over cyclooxygenase-1 inhibition of at least 10, and more preferably of at least 40. The resulting selectivity may indicate an ability to reduce the incidence of common NSAID-induced side effects.

“Inhibitor of cyclooxygenase-2”, “cyclooxygenase-2 inhibitor” and “COX-2 inhibitor” as used herein embrace compounds which selectively inhibit cyclooxygenase-2 over cyclooxygenase-1.

The inhibitor of cyclooxygenase-2 may be administered at a dosage level up to conventional dosage levels for NSAIDs. Suitable dosage levels will depend upon the anti-inflammatory effect of the chosen inhibitor of cyclooxygenase-2, but typically suitable levels will be about 0.001 to 50 mg/kg per day, preferably 0.005 to 30 mg/kg per day, and especially 0.05 to 10 mg/kg per day. The compound may be administered on a regimen of up to 6 times per day, preferably 1 to 4 times per day, and especially once per day.

As explained in J. Talley, Exp. Opin. Ther. Patents (1997), 7(1), pp. 55-62, three distinct structural classes of selective COX-2 inhibitor compounds have been identified. One class is the methane sulfonanilide class of inhibitors, of which NS-398, flosulide, nimesulide and (i) are example members.

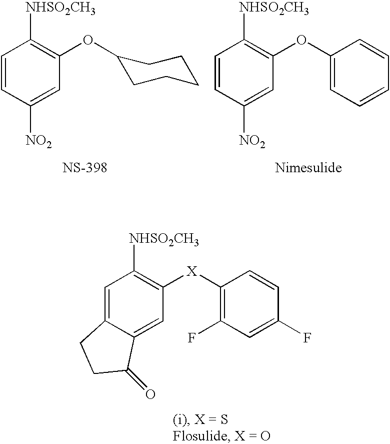

A second class is the tricyclic inhibitor class, which can be further divided into the sub-classes of tricyclic inhibitors with a central carbocyclic ring (examples include SC-57666, 1, and 2); those with a central monocyclic heterocyclic ring (examples include DuP 697, SC-58125, SC-58635, and 3, 4 and 5; and those with a central bicyclic heterocyclic ring (examples include 6, 7, 8, 9 and 10). Compounds 3, 4 and 5 are described in U.S. Pat. No. 5,474,995.

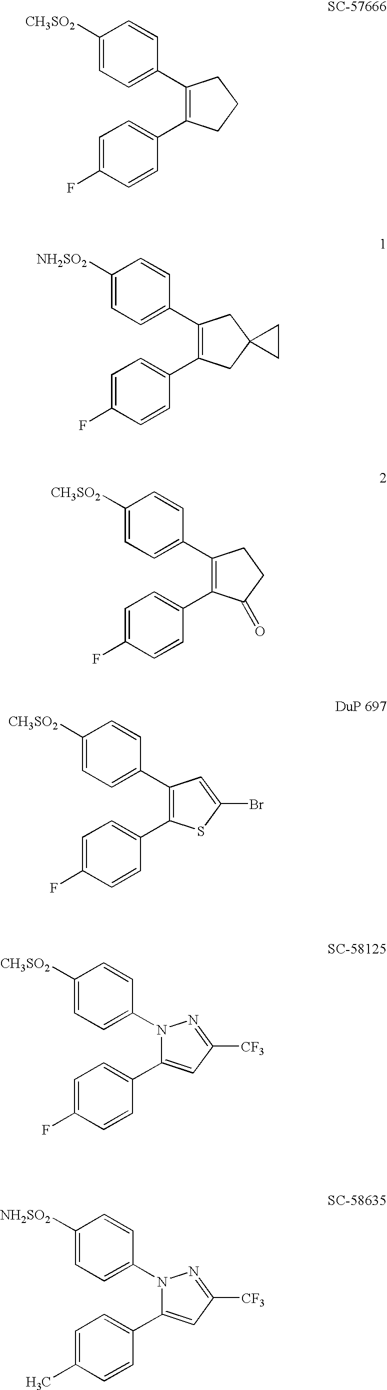

Also within this class are compounds described in U.S. Pat. No. 5,861,419, e.g., 5-chloro-3-(4-methanesulfonylphenyl)-6′-methyl-[2,3′]bipyridinyl. The third identified class can be referred to as those which are structurally modified NSA/Ds, and includes 11a and structure 11 as example members.

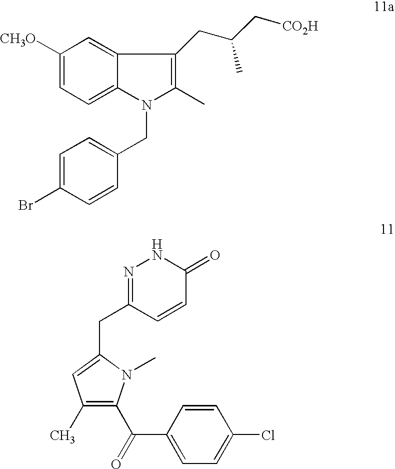

In addition to the structural classes, sub-classes, specific COX-2 inhibitor compound examples, and reference journal and patent publications described in the Talley publication which are all herein incorporated by reference, examples of compounds which selectively inhibit cyclooxygenase-2 have also been described in the following patent publications, all of which are herein incorporated by reference: U.S. Pat. Nos. 5,344,991, 5,380,738, 5,393,790, 5,409,944, 5,434,178, 5,436,265, 5,466,823, 5,474,995, 5,510,368, 5,536,752, 5,550,142, 5,552,422, 5,604,253, 5,604,260, 5,639,780; and International Patent Specification Nos. 94/13635, 94/15932, 94/20480, 94/26731, 94/27980, 95/00501, 95/15316, 96/03387, 96/03388, 96/06840; and International Publication No.'s WO 94/20480, WO 96/21667, WO 96/31509, WO 96/36623, WO 97/14691, WO 97/16435.

Additional COX-2 inhibitor compounds which are included in the scope of this invention include:

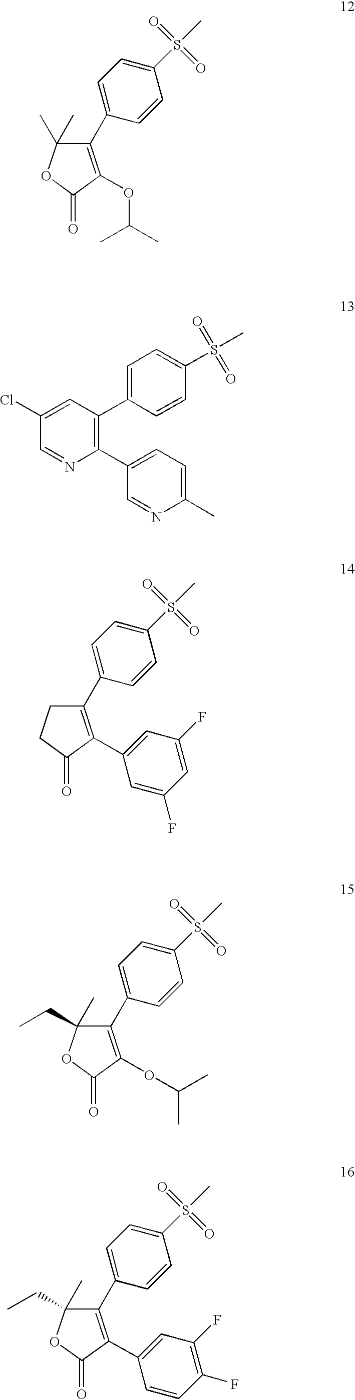

Some of the compounds above can also be identified by the following chemical names:

3: 3-phenyl-4-(4-(methylsulfonyl)phenyl)-2-(5H)-furanone;

4: 3-(3,4-difluorophenyl)-4-(4-(methylsulfonyl)phenyl)-2-(5H)-furanone;

5: 5,5-dimethyl-4-(4-(methylsulfonyl)phenyl)-3-(3-fluorophenyl)-5H-furan-2-one;

12: 5,5-dimethyl-4-(4-(methylsulfonyl)phenyl)-3-(2-propoxy)-5H-furan-2-one

13: 5-chloro-3-(4-(methylsulfonyl)phenyl)-2-(2-methyl-5-pyridinyl)pyridine

14: 2-(3,5-difluorophenyl)-3-(4-(methylsulfonyl)phenyl)-2-cyclopenten-1-one

15: 5(S)-5-ethyl-5-methyl-4-(4-(methylsulfonyl)phenyl)-3-(2-propoxy)-5H-furan-2-one

16: 5-ethyl-5-methyl-4-(4-(methylsulfonyl)phenyl)-3-(3,4-difluorophenyl)-5H-furan-2-one;

17: 3-((2-thiazolyl)methoxy)-4-(4-(methylsulfonyl)phenyl)-5,5-dimethyl-5H-furan-2-one

18: 3-propyloxy-4-(4-(methylsulfonyl)phenyl)-5,5-dimethyl-5H-furan-2-one

19: 3-(1-cyclopropylethoxy)-5,5-dimethyl-4-(4-methylsulfonyl)phenyl)-5H-furan-2-one

20: sodium 2-(4-chlorophenyl)-3-(4-(methylsulfonyl)phenyl)-4-oxo-2-pentenoate;

21: 3-(cyclopropylmethoxy)-5,5-dimethyl-4-(4-(methylsulfonyl)phenyl)-5H-furan-2-one

22: 3-(cyclopropylmethoxy)-5,5-dimethyl-4-(4-(methylsulfonyl)phenyl)-2,5-dihydrofuran-2-ol;

23: 3-isopropoxy-5,5-dimethyl-4-(4-(methylsulfonyl)phenyl)-2,5-dihydrofuran-2-ol

24: 5,5-dimethyl-3-(3-fluorophenyl)-2-hydroxy-4-(4-(methylsulfonyl)phenyl)-2,5-dihydrofuran

25: 5-Chloro-3-(4-(methylsulfonyl)phenyl)-2-(3-pyridinyl)pyridine

The following publications describe and/or provide methods for making the compounds as indicated: compounds 12, 15, 17, 18, 19 and 21, WO 97/14691; compounds 22, 23 and 24, WO 97/16435; compound 20, WO 96/36623; compound 14, U.S. Pat. No. 5,536,752; compound 16, U.S. Pat. No. 5,474,995. See Examples herein for compounds 13 and 25.

Also incorporated herein by reference are those compounds described in WO 96/41645 as having structural Formula I, shown below, and the definition and preferred definitions and species described therein:

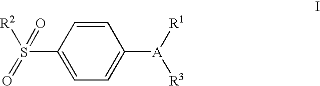

Particularly preferred compounds of formula (I) include:

5-(4-fluorophenyl)-1-[4-(methylsulfonyl)phenyl]-3-(trifluoromethyl)pyrazole;

4-(4-fluorophenyl)-5-[4-(methylsulfonyl)phenyl]-1-phenyl-3-(trifluoromethyl)pyrazole;

4-(5-(4-chlorophenyl)-3-(4-methoxyphenyl)-1H-pyrazol-1-yl)benzenesulfonamide;

4-(3,5-bis(4-methylphenyl)-1H-pyrazol-1-yl)benzenesulfonamide;

4-(5-(4-chlorophenyl)-3-phenyl-1H-pyrazol-1-yl)benzenesulfonamide;

4-(3,5-bis(4-methoxyphenyl)-1H-pyrazol-1-yl)benzenesulfonamide;

4-(5-(4-chlorophenyl)-3-(4-methylphenyl)-1H-pyrazol-1-yl)benzenesulfonamide;

4-(5-(4-chlorophenyl)-3-(4-nitrophenyl)-1H-pyrazol-1-yl)benzenesulfonamide;

4-(5-(4-chlorophenyl)-3-(5-chloro-2-thienyl)-1H-pyrazol-1-yl)benzenesulfonamide;

4-(4-chloro-3,5-diphenyl-1H-pyrazol-1-yl)benzenesulfonamide;

4-(5-(4-chlorophenyl)-3-(trifluoromethyl)-1H-pyrazol-1-yl)benzenesulfonamide;

4-(5-phenyl)-3-(trifluoromethyl)-1H-pyrazol-1-yl)benzenesulfonamide;

4-(5-(4-fluorophenyl)-3-(trifluoromethyl)—H-pyrazol-1-yl)benzenesulfonamide;

4-(5-(4-methoxyphenyl)-3-(trifluoromethyl)-1H-pyrazol-1-yl)benzenesulfonamide;

4-(5-(4-chlorophenyl)-3-(difluoromethyl)-1H-pyrazol-1-yl)benzenesulfonamide;

4-(5-(4-methylphenyl)-3-(trifluoromethyl)-1H-pyrazol-1-yl)benzenesulfonamide;

4-(4-chloro-5-(4-chlorophenyl)-3-(trifluoromethyl)-1H-pyrazol-1-yl)benzenesulfonamide;

4-(3-(difluoromethyl)-5-(4-methylphenyl) 1H-pyrazol-1-yl)benzenesulfonamide;

4-(3-ifluoromethyl)-5-phenyl-1H-pyrazol-1-yl)benzenesulfonamide;

4-(3-(difluoromethyl)-5-(4-methoxyphenyl)-1H-pyrazol-1-yl)benzenesulfonamide;

4-(3-cyano-5-(4-fluorophenyl)-1H-pyrazol-1-yl)benzenesulfonamide;

4-(3-(difluoromethyl)-5-(3-fluoro-4-methoxyphenyl)-1H-pyrazol-1-yl)benzenesulfonamide;

4-(5-(3-fluoro-4-methoxyphenyl)-3-(trifluoromethyl)-1H-pyrazol-1-yl)benzenesulfonamide;

4-(4-chloro-5-phenyl-1H-pyrazol-1-yl)benzenesulfonamide;

4-(5-(4-chlorophenyl)-3-(hydroxyphenyl)-1H-pyrazol-1-yl)benzenesulfonamide;

4-(5-(4-(N,N-dimethylamino)phenyl)-3-(trifluoromethyl)-1H-pyrazol-1-yl)benzenesulfonamide;

5-(4-fluorophenyl)-6-(4-(methylsulfonyl)phenyl)spiro[2.4]hept-5-ene;

4-(6-(4-fluorophenyl)spiro[2.4]hept-5-en-5-yl)benzenesulfonamide;

6-(4-fluorophenyl)-7-(4-(methylsulfonyl)phenyl)spiro[3.4]oct-6-ene;

5-(3-chloro-4-methoxyphenyl)-6-(4-(methylsulfonyl)phenyl)spiro[2.4]hept-5-ene;

4-(6-(3-chloro-4-methoxyphenyl)spiro[2.4]hept-5-en-5-yl)benzenesulfonamide;

5-(3,5-dichloro-4-methoxyphenyl)-6-(4-(methylsulfonyl)phenyl)spiro[2.4]hept-5-ene;

5-(3-chloro-4-fluorophenyl)-6-(4-(methylsulfonyl)phenyl)spiro[2.4]hept-5-ene;

4-(6-(3,4-dichlorophenyl)spiro[2.4]hept-5-en-5-yl)benzenesulfonamide;

2-(3-chloro-4-fluorophenyl)-4-(4-fluorophenyl)-5-(4-methylsulfonylphenyl)thiazole;

2-(2-chlorophenyl)-4-(4-fluorophenyl)-5-(4-methylsulfonylphenyl)thiazole;

5-(4-fluorophenyl)-4-(4-methylsulfonylphenyl)-2-methylthiazole;

4-(4-fluorophenyl)-5-(4-methylsulfonylphenyl)-2-trifluoromethylthiazole;

4-(4-fluorophenyl)-5-(4-methylsulfonylphenyl)-2-(2-thienyl)thiazole;

4-(4-fluorophenyl)-5-(4-methylsulfonylphenyl)-2-benzylaminothiazole;

4-(4-fluorophenyl)-5-(4-methylsulfonylphenyl)-2-(1-propylamino)thiazole;

2-((3,5-dichlorophenoxy)methyl)-4-(4-fluorophenyl)-5-(4-(methylsulfonyl)phenyl)thiazole;

5-(4-fluorophenyl)-4-(4-methylsulfonylphenyl)-2-trifluoromethylthiazole;

1-methylsulfonyl-4-(1,1-dimethyl-4-(4-fluorophenyl)cyclopenta-2,4-dien-3-yl)benzene;

4-(4-(4-fluorophenyl)-1,1-dimethylcyclopenta-2,4-dien-3-yl)benzenesulfonamide;

5-(4-fluorophenyl)-6-(4-(methylsulfonyl)phenyl)spiro[2.4]hepta-4,6-diene;

4-(6-(4-fluorophenyl)spiro[2.4]hepta-4,6-dien-5-yl)benzenesulfonamide;

6-(4-fluorophenyl)-2-methoxy-5-(4-(methylsulfonyl)phenyl)-pyridine-3-carbonitrile;

2-bromo-6-(4-fluorophenyl)-5-(4-(methylsulfonyl)phenyl)-pyridine-3-carbonitrile;

6-(4-fluorophenyl)-5-(4-(methylsulfonyl)phenyl)-2-phenyl-pyridine-3-c arbonitrile;

4-(2-(4-methylpyridin-2-yl)-4-(trifluoromethyl)-1H-imidazol-1-yl)benzenesulfonamide;

4-(2-(5-methylpyridin-3-yl)-4-(trifluoromethyl)-H-imidazol-1-yl)benzenesulfonamide;

4-(2-(2-methylpyridin-3-yl)-4-(trifluoromethyl)-1H-imidazol-1-yl)benzenesulfonamide;

3-(1-(4-(methylsulfonyl)phenyl)-4-(trifluoromethyl)-1H-imidazol-2-yl)benzenesulfonamide;

2-(1-(4-(methylsulfonyl)phenyl)-4-(trifluoromethyl)-1H-imidazol-2-yl)pyridine;

2-methyl-4-(1-(4-(methylsulfonyl)phenyl)-4-(trifluoromethyl)-1H-imidazol-2-yl)pyridine;

2-methyl-6-(1-(4-(methylsulfonyl)phenyl)-4-(trifluoromethyl)-1H-imidazol-2-yl)pyridine;

4-(2-(6-methylpyridin-3-yl)-4-(trifluoromethyl)-1H-imidazol-1-yl)benzenesulfonamide;

2-(3,4-difluorophenyl)-1-(4-(methylsulfonyl)phenyl)-4-(trifluoromethyl)-1H-imidazole;

4-(2-(4-methylphenyl)-4-(trifluoromethyl)-1H-imidazol-1-yl)benzenesulfonamide;

2-(4-chlorophenyl)-1-(4-(methylsulfonyl)phenyl)-4-methyl-1H-imidazole;

2-(4-chlorophenyl)-1-(4-(methylsulfonyl)phenyl)-4-phenyl-1H-imidazole;

2-(4-chlorophenyl)-4-(4-fluorophenyl)-1-(4-(methylsulfonyl)phenyl)-1H-imidazole;

2-(3-fluoro-4-methoxyphenyl)-1-(4-(methylsulfonyl)phenyl)-4-(trifluoromethyl)-1H-imidazole;

1-(4-(methylsulfonyl)phenyl)-2-phenyl-4-trifluoromethyl-1H-imidazole;

2-(4-methylphenyl)-1-(4-(methylsulfonyl)phenyl)-4-trifluoromethyl-1H-imidazole;

4-(2-(3-chloro-4-methylphenyl)-4-(trifluoromethyl)-1H-imidazol-1-yl)benzenesulfonamide;

2-(3-fluoro-5-methylphenyl)-1-(4-(methylsulfonyl)phenyl)-4-(trifluoromethyl)-1H-imidazole;

4-(2-(3-fluoro-5-methylphenyl)-4-(trifluoromethyl)-1H-imidazol-1-yl)benzenesulfonamide;

2-(3-methylphenyl)-1-(4-(methylsulfonyl)phenyl)-4-(trifluoromethyl)-1H-imidazole;

4-(2-(3-methylphenyl)-4-(trifluoromethyl)-1H-imidazol-1-yl)benzenesulfonamide;

1-(4-(methylsulfonyl)phenyl)-2-(3-chlorophenyl)-4-(trifluoromethyl)-1H-imidazole; 4-(2-(3-chlorophenyl)-4-(trifluoromethyl)-1H-imidazol-1-yl)benzenesulfonamide;

4-(2-phenyl-4-(trifluoromethyl)-1H-imidazol-1-yl)benzenesulfonamide;

4-(2-(4-methoxy-3-chlorophenyl)-4-(trifluoromethyl)-1H-imidazol-1-yl)benzenesulfonamide;

1-allyl-4-(4-fluorophenyl)-3-(4-(methylsulfonyl)phenyl)-5-(trifluoromethyl)-1H-pyrazole;

4-(1-ethyl-4-(4-fluorophenyl)-5-(trifluoromethyl)-1H-pyrazol-3-yl)benzenesulfonamide;

N-phenyl-(4-(4-fluorophenyl)-3-(4-(methylsulfonyl)phenyl)-5-(trifluoromethyl)-1H-pyrazol-1-yl)acetamide;

ethyl (4-(4-fluorophenyl)-3-(4-(methylsulfonyl)phenyl)-5-(trifluoromethyl)-1H-pyrazol-1-yl)acetate;

4-(4-fluorophenyl)-3-(4-(methylsulfonyl)phenyl)-1-(2-phenylethyl)-1H-pyrazole;

4-(4-fluorophenyl)-3-(4-(methylsulfonyl)phenyl)-1-(2-phenylethyl)-5-(trifluoromethyl)pyrazole;

1-ethyl-4-(4-fluorophenyl)-3-(4-(methylsulfonyl)phenyl)-5-(trifluoromethyl)-H-pyrazole;

5-(4-fluorophenyl)-4-(4-(methylsulfonyl)phenyl)-2-(trifluoromethyl)-1H-imidazole;

4-(4-(methylsulfonyl)phenyl)-5-(2-thiophenyl)-2-(trifluoromethyl)-1H-imidazole;

5-(4-fluorophenyl)-2-methoxy-4-(4-(methylsulfonyl)phenyl)-6-(trifluoromethyl)pyridine;

2-ethoxy-5-(4-fluorophenyl)-4-(4-(methylsulfonyl)phenyl)-6-(trifluoromethyl)pyridine;

5-(4-fluorophenyl)-4-(4-(methylsulfonyl)phenyl)-2-(2-propynyloxy)-6-(trifluoromethyl)pyridine;

2-bromo-5-(4-fluorophenyl)-4-(4-(methylsulfonyl)phenyl)-6-(trifluoromethyl)pyridine;

4-(2-(3-chloro-4-methoxyphenyl)-4,5-difluorophenyl)benzenesulfonamide;

1-(4-fluorophenyl)-2-(4-(methylsulfonyl)phenyl)benzene;

5-difluoromethyl-4-(4-(methylsulfonyl)phenyl)-3-phenylisoxazole;

4-(3-ethyl-5-phenylisoxazol-4-yl)benzenesulfonamide;

4-(5-difluoromethyl-3-phenylisoxazol-4-yl)benzenesulfonamide;

4-(5-hydroxymethyl-3-phenylisoxazol-4-yl)benzenesulfonamide;

4-(5-methyl-3-phenylisoxazol-4-yl) benzenesulfonamide;

1-(2-(4-fluorophenyl)cyclopenten-1-yl)-4-(methylsulfonyl)benzene;

1-(2-(4-fluoro-2-methylphenyl)cyclopenten-1-yl)-4-(methylsulfonyl)benzene;

1-(2-(4-chloro phenyl)cyclopenten-1-yl)-4-(methylsulfonyl)benzene;

1-(2-(2,4-dichlorophenyl)cyclopenten-1-yl)-4-(methylsulfonyl)benzene;

1-(2-(4-trifluoromethylphenyl)cyclopenten-1-yl)-4-(methylsulfonyl)benzene;

1-(2-(4-methylthiophenyl)cyclopenten-1-yl)-4-(methylsulfonyl)benzene;

1-(2-(4-fluorophenyl)4,4-dimethylcyclopenten-1-yl)-4-(methylsulfonyl)benzene;

4-(2-(4-fluorophenyl)-4,4-dimethylcyclopenten-1-yl)benzenesulfonamide;

1-(2-(4-chlorophenyl)-4,4-dimethylcyclopenten-1-yl)-4-(methylsulfonyl)benzene;

4-(2-(4-chlorophenyl)-4,4-dimethylcyclopenten-1-yl)benzenesulfonamide;

4-(2-(4-fluorophenyl)cyclopenten-1-yl)benzenesulfonamide;

4-(2-(4-chlorophenyl)cyclopenten-1-yl)benzenesulfonamide;

1-(2-(4-methoxyphenyl)cyclopenten-1-yl)-4-(methylsulfonyl)benzene;

1-(2-(2,3-difluorophenyl)cyclopenten-1-yl)-4-(methylsulfonyl)benzene;

4-(2-(3-fluoro-4-methoxyphenyl)cyclopenten-1-yl)benzenesulfonamide;

1-(2-(3-chloro-4-methoxyphenyl)cyclopenten-1-yl)-4-(methylsulfonyl)benzene;

4-(2-(3-chloro-4-fluorophenyl)cyclopenten-1-yl)benzenesulfonamide;

4-(2-(2-methylpyridin-5-yl)cyclopenten-1-yl)benzenesulfonamide;

ethyl 2-(4-(4-fluorophenyl)-5-(4-(methylsulfonyl)phenyl)oxazol-2-yl)-2-benzyl-acetate;

2-(4-(4-fluorophenyl)-5-(4-(methylsulfonyl)phenyl)oxazol-2-yl)acetic acid;

2-(tert-butyl)-4-(4-fluorophenyl)-5-(4-(methylsulfonyl)phenyl)oxazole;

4-(4-fluorophenyl)-5-(4-(methylsulfonyl)phenyl)-2-phenyloxazole;

4-(4-fluorophenyl)-2-methyl-5-(4-(methylsulfonyl)phenyl)oxazole;

4-(5-(3-fluoro-4-methoxyphenyl)-2-trifluoromethyl-4-oxazolyl)benzenesulfonamide; and

5-chloro-3-(4-methanesulfonylphenyl)-6′-methyl-[2,3 ]bipyridinyl,

or a pharmaceutically acceptable salt thereof.

The dosage regimen utilizing an inhibitor of activated thrombin activatable fibrinolysis inhibitor in combination with the NSAID is selected in accordance with a variety of factors including type, species, age, weight, sex and medical condition of the patient; the severity of the condition to be treated; the route of administration; the renal and hepatic function of the patient; and the particular compound or salt or ester thereof employed. Since two different active agents are being used together in a combination therapy, the potency of each of the agents and the interactive effects achieved by combining them together must also be taken into account. A consideration of these factors is well within the purview of the ordinarily skilled clinician for the purpose of determining the therapeutically effective or prophylactically effective dosage amounts needed to prevent, counter, or arrest the progress of the condition.

Administration of the drug combination to the patient includes both self-administration and administration to the patient by another person.

Additional active agents may be used in combination with the NSAID and inhibitor of activated thrombin activatable fibrinolysis inhibitor in a single dosage formulation, or may be administered to the patient in a separate dosage formulation, which allows for concurrent or sequential administration. Examples of additional active agents which may be employed include HMG-CoA synthase inhibitors; squalene epoxidase inhibitors; squalene synthetase inhibitors (also known as squalene synthase inhibitors), acyl-coenzyme A: cholesterol acyltransferase (ACAT) inhibitors; probucol; niacin; fibrates such as clofibrate, fenofibrate, and gemfibrizol; cholesterol absorption inhibitors; bile acid sequestrants; LDL (low density lipoprotein) receptor inducers; vitamin B₆ (also known as pyridoxine) and the pharmaceutically acceptable salts thereof such as the HCl salt; vitamin B₁₂ (also known as cyanocobalamin); β-adrenergicreceptor blockers; folic acid or a pharmaceutically acceptable salt or ester thereof such as the sodium salt and the methylglucamine salt; and anti-oxidant vitamins such as vitamin C and E and beta carotene.

The active drugs can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The active drugs may also be delivered by the use of monoclonal antibodies as individual carriers to which the compound molecules are coupled. They may also be coupled with soluble polymers as targetable drug carriers. Such polymers can include polyinyl-pyrrolidone, pyran copolymer, polyhydroxy-propyl-methacrylamide-phenol, polyhydroxy-ethyl-aspartamide-phenol, or polyethyleneoxide-polylysine substituted with palmitoyl residues. Furthermore, the active drugs may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels.

Although the active agents may be administered in divided doses, for example two or three times daily, a single daily dose of each of the inhibitor of activated thrombin activatable fibrinolysis inhibitor and the NSAID is preferred, with a single daily dose of both agents in a single pharmaceutical composition being most preferred.

An additional embodiment of the instant invention involves a kit comprised of an NSAID such as a COX-2 inhibitor in an oral dosage formulation and an inhibitor of activated thrombin activatable fibrinolysis inhibitor in a separate oral dosage formulation. More particularly, the kit is comprised of a COX-2 inhibitor selected from the group consisting of 5-chloro-3-(4-(methylsulfonyl)phenyl)-2-(2-methyl-5-pyridinyl)pyridine; 2-(3,5-difluorophenyl)-3-(4-(methylsulfonyl)phenyl)-2-cyclopenten-1-one; 3-phenyl-4-(4-(methylsulfonyl)phenyl)-2-(5H)-furanone; 3-(3,4-difluorophenyl)-4-(4-(methylsulfonyl)phenyl)-2-(5H)-furanone; 5,5-dimethyl-4-(4-(methylsulfonyl)phenyl)-3-(3-fluorophenyl)-5H-furan-2-one, and 5-chloro-3-(4-methanesulfonylphenyl)-6′-methyl-[2,3]bipyridinyl; and the inhibitor of activated thrombin activatable fibrinolysis inhibitor is selected from the group consisting of 2-(6-amino-pyridin-3-ylmethyl)-3-butyl-succinic acid, 2-(6-amino-pyridin-3-ylmethyl)-3-phenethyl-succinic acid, 2-(6-amino-pyridin-3-ylmethyl)-3-methyl-succinic acid, 2-(6-amino-5-methyl-pyridin-3-ylmethyl)-3-[(1-benzyloxycarbonylamino-2-methyl-propyl)hydroxy-phosphinoyl]-propionic acid, 2-(6-amino-pyridin-3-ylmethyl)-3-[hydroxy-(3-phenyl-propyl)-phosphinoyl]-propionic acid, 2-(amino-pyridin-3-ylmethyl)—N-hydroxy-succinamic acid, 3-(6-amino-pyridin-3-yl)-2-mercaptomethyl-propionic acid, 2-(2-amino-pyridin-4-ylmethyl)-3-mercapto-propionic acid, 2-(6-amino-pyridin-3-ylmethyl)-2-mercaptomethyl-butyric acid, 3-(6-amino-5-methyl-pyridin-3-yl)-2-mercaptomethyl-2-methyl-propionic acid, 3-(6-amino-5-methyl-pyridin-3-yl)-2-mercapto methyl-propionic acid, 3-(6-amino-4-methyl-pyridin-3-yl)-2-mercaptomethyl-propionic acid, and 3-(6-amino-pyridin-3-yl)-2-mercaptomethyl-butyric acid. In one class of this embodiment the COX-2 inhibitor is selected from 5-chloro-3-(4-(methylsulfonyl)phenyl)-2-(2-methyl-5-pyridinyl)pyridine; 2-(3,5-difluorophenyl)-3-(4-(methylsulfonyl)phenyl)-2-cyclopenten-1-one; 3-phenyl-4-(4-(methylsulfonyl)phenyl)-2-(5H)-furanone; 3-(3,4-difluorophenyl)-4-(4-(methylsulfonyl)phenyl)-2-(5H)-furanone; 5,5-dimethyl-4-(4-(methylsulfonyl)phenyl)-3-(3-fluorophenyl)-5H-furan-2-one, and 5-chloro-3-(4-methanesulfonylphenyl)-6′-methyl-[2,3]bipyridinyl.

One example of this embodiment is a kit comprised of an oral dosage formulation of a COX-2 inhibitor and an oral dosage formulation of an inhibitor of activated thrombin activatable fibrinolysis inhibitor. The packaging for the kit could be designed and manufactured in a variety of ways. A preferred example is a blister package containing rows of a COX-2 inhibitor tablet and an inhibitor of activated thrombin activatable fibrinolysis inhibitor tablet placed side by side on the same blister card, each of the two tablets in its own blister bubble, with calendar or similar type markings on the card that convey to the user that one “pair” of tablets (i.e., one COX-2 inhibitor tablet and one inhibitor of activated thrombin activatable fibrinolysis inhibitor tablet) is to be ingested per day.

The examples are merely illustrative and should not be interpreted as limiting the scope of the claimed invention.

### EXAMPLE 1

Tablet Preparation

Exemplary inhibitors of activated thrombin activatable fibrinolysis inhibitor tablet compositions include those shown below where the inhibitors of activated thrombin activatable fibrinolysis inhibitor is 3-(6-amino-pyridin-3-yl)-2-mercaptomethyl-propionic acid:

| Component | 0.25 mg | 2 mg | 10 mg | 50 mg |
|---|---|---|---|---|
| inhibitor of activated | 0.500% | 1.000% | 5.000% | 14.29% |
| thrombin activatable | | | | |
| fibrinolysis inhibitor | | | | |
| mannitol | 49.50 | 49.25 | 47.25 | 42.61 |
| microcrystalline cellulose | 49.50 | 49.25 | 47.25 | 42.61 |
| magnesium stearate | 0.500 | 0.500 | 0.50 | 0.500 |

2, 10 and 50 mg tablets were film-coated with an aqueous dispersion of hydroxypropyl cellulose, hydroxypropyl methylcellulose and titanium dioxide, providing a nominal weight gain of 2.4%.

Tablet Preparation Via Direct Compression

The inhibitors of activated thrombin activatable fibrinolysis inhibitor, mannitol and microcrystalline cellulose were sieved through mesh screens of specified size (generally 250 to 750 μm) and combined in a suitable blender. The mixture was subsequently blended (typically 15 to 30 min) until the drug was uniformly distributed in the resulting dry powder blend. Magnesium stearate was screened and added to the blender, after which a precompression tablet blend was achieved upon additional mixing (typically 2 to 10 min). The precompression tablet blend was then compacted under an applied force, typically ranging from 0.5 to 2.5 metric tons, sufficient to yield tablets of suitable physical strength with acceptable disintegration times (specifications will vary with the size and potency of the compressed tablet). In the case of the 2, 10 and 50 mg potencies, the tablets were dedusted and film-coated with an aqueous dispersion of water-soluble polymers and pigment.

Tablet Preparation Via Dry Granulation

Alternatively, a dry powder blend is compacted under modest forces and remilled to afford granules of specified particle size. The granules are then mixed with magnesium stearate and tabletted as stated above.

### EXAMPLE 2

Intravenous Formulations

Exemplary inhibitor of activated thrombin activatable fibrinolysis inhibitor intravenous formulations, prepared according to general intravenous formulation procedures, are those shown below where the inhibitors of activated thrombin activatable fibrinolysis inhibitor 3-(6-amino-pyridin-3-yl)-2-mercaptomethyl-propionic acid:

| Component | Estimated range |
|---|---|
| Inhibitor of activated thrombin | 0.1-1.2 mg |
| activatable fibrinolysis inhibitor | |
| D-glucuronic acid | 0.5-5 mg |
| Mannitol NE | 50-53 mg |
| Water for injection | q.s. 1.0 mL |

1N sodium hydroxide is used to achieve a solution pH in the range of between 3.9-4.1.

Exemplary compositions A-C are as follows:

| Component | A | B | C |
|---|---|---|---|
| Inhibitor of activated | 1.0 mg | 0.50 | 0.12 |
| thrombin activatable | | | |
| fibrinolysis inhibitor | | | |
| D-glucuronic acid | 1.94 mg | 1.94 mg | 1.94 mg |
| Mannitol NF | 51.2 mg | 51.2 mg | 51.2 mg |
| 1 N Sodium Hydroxide | q.s. pH 4.0 | q.s. pH 4.0 | q.s. pH 4.0 |
| Water for injection | q.s. 1.0 mL | q.s. 1.0 mL | q.s. 1.0 mL |

Various other buffer acids, such as L-lactic acid, acetic acid, citric acid or any pharmaceutically acceptable acid/conjugate base with reasonable buffering capacity in the pH range acceptable for intravenous administration may be substituted for glucuronic acid.

### EXAMPLE 3

Subcutaneous Formulations

Exemplary inhibitors of activated thrombin activatable fibrinolysis inhibitor subcutaneous formulations, prepared according to general subcutaneous formulation procedures, are those shown below where the inhibitor of activated thrombin activatable fibrinolysis inhibitor is 3-(6-amino-pyridin-3-yl)-2-mercaptomethyl-propionic acid:

Component

| | |
|---|---|
| Inhibitor of activated thrombin | 25 mg/ml |
| activatable fibrinolysis inhibitor | |
| Citric acid buffer | 10 mM |
| Sodium chloride | 5 mg/ml |
| Water for injection | q.s. 1.0 ml |

1N sodium hydroxide is used to achieve a solution pH in the range of between 7.0-7.4.

Examples of dosage formulations suitable for use in practicing the instant invention follow. In the examples, the COX-2 inhibitor is 3-phenyl-4-(4-(methylsulfonyl)phenyl)-2-(5H)-furanone (except for Example 10 where the COX-2 inhibitor is 3-(3,4-difluorophenyl)-4-(4-(methylsulfonyl)phenyl)-2-(5H)-furanone) and the inhibitor of activated thrombin activatable fibrinolysis inhibitor is 3-(6-amino-pyridin-3-yl)-2-mercaptomethyl-propionic acid. Alternatively, the examples are prepared wherein the COX-2 inhibitor is 5-chloro-3-(4-methanesulfonylphenyl)-6′-methyl-[2,3 ]bipyridinyl.

### EXAMPLE 4

Wet granulated tablet composition

| Amount per tablet | | Ingredient |
|---|---|---|
| 25 | mg | COX-2 inhibitor |
| 79.7 | mg | Microcrystalline cellulose |
| 79.7 | mg | Lactose monohydrate |
| 6 | mg | Hydroxypropyl cellulose |
| 8 | mg | Croscarmellose sodium |
| 0.6 | mg | Iron oxide |
| 1 | mg | Magnesium stearate |

Tablet dose strengths of between 5 and 125 mg can be accommodated by varying total tablet weight, and the ratio of the first three ingredients. Generally it is preferable to maintain a 1:1 ratio for microcrystalline cellulose lactose monohydrate.

### EXAMPLE 4A

Wet granulated tablet composition

| Amount per tablet | | Ingredient |
|---|---|---|
| 12.5 | mg | COX-2 inhibitor |
| 86 | mg | Microcrystalline cellulose |
| 86 | mg | Lactose monohydrate |
| 6 | mg | Hydroxypropyl cellulose |
| 8 | mg | Croscarmellose sodium |
| 0.6 | mg | Iron oxide |
| 1 | mg | Magnesium stearate |

### EXAMPLE 4B

Wet granulated tablet composition

| Amount per tablet | | Ingredient |
|---|---|---|
| 10 | mg | COX-2 inhibitor |
| 87.2 | mg | Microcrystalline cellulose |
| 87.2 | mg | Lactose monohydrate |
| 6 | mg | Hydroxypropyl cellulose |
| 8 | mg | Croscarmellose sodium |
| 0.6 | mg | Iron oxide |
| 1 | mg | Magnesium stearate |

### EXAMPLE 4C

Wet granulated tablet composition

| Amount per tablet | | Ingredient |
|---|---|---|
| 5 | mg | COX-2 inhibitor |
| 89.7 | mg | Microcrystalline cellulose |
| 89.7 | mg | Lactose monohydrate |
| 6 | mg | Hydroxypropyl cellulose |
| 8 | mg | Croscarmellose sodium |
| 0.6 | mg | Iron oxide |
| 1 | mg | Magnesium stearate |

### EXAMPLE 5

Directly compressed tablet composition

| Amount per tablet | | Ingredient |
|---|---|---|
| 25 | mg | COX-2 inhibitor |
| 106.9 | mg | Microcrystalline cellulose |
| 106.9 | mg | Lactose anhydrate |
| 7.5 | mg | Croscarmellose sodium |
| 3.7 | mg | Magnesium stearate |

Tablet dose strengths of between 5 and 125 mg can be accommodated by varying total tablet weight, and the ratio of the first three ingredients. Generally it is preferable to maintain a 1:1 ratio for microcrystalline cellulose: lactose monohydrate.

### EXAMPLE 5A

Directly compressed tablet composition

| Amount per tablet | | Ingredient |
|---|---|---|
| 12.5 | mg | COX-2 inhibitor |
| 113.2 | mg | Microcrystalline cellulose |
| 113.2 | mg | Lactose anhydrate |
| 7.5 | mg | Croscarmellose sodium |
| 3.7 | mg | Magnesium stearate |

### EXAMPLE 5B

Directly compressed tablet composition

| Amount per tablet | | Ingredient |
|---|---|---|
| 10 | mg | COX-2 inhibitor |
| 42.5 | mg | Microcrystalline cellulose |
| 42.5 | mg | Lactose anhydrate |
| 4 | mg | Croscarmellose sodium |
| 1 | mg | Magnesium stearate |

### EXAMPLE 5C

Directly compressed tablet composition

| Amount per tablet | | Ingredient |
|---|---|---|
| 5 | mg | COX-2 inhibitor |
| 45 | mg | Microcrystalline cellulose |
| 45 | mg | Lactose anhydrate |
| 4 | mg | Croscarmellose sodium |
| 1 | mg | Magnesium stearate |

### EXAMPLE 6

Hard gelatin capsule composition

| Amount per capsule | | Ingredient |
|---|---|---|
| 25 | mg | COX-2 inhibitor |
| 37 | mg | Microcrystalline cellulose |
| 37 | mg | Lactose anhydrate |
| 1 | mg | Magnesium stearate |
| 1 | capsule | Hard gelatin capsule |

Capsule dose strengths of between 1 and 50 mg can be accommodated by varying total fill weight, and the ratio of the first three ingredients. Generally it is preferable to maintain a 1:1 ratio for microcrystalline cellulose: lactose monohydrate.

### EXAMPLE 7

Oral solution

| Amount per 5 mL dose | | Ingredient |
|---|---|---|
| 50 | mg | COX-2 inhibitor |
| to 5 | mL with Polyethylene oxide 400 | |

Solution dose strengths of between 1 and 50 mg/5 mL can be accommodated by varying the ratio of the two ingredients.

### EXAMPLE 8

Oral suspension

| Amount per 5 mL dose | | Ingredient |
|---|---|---|
| 101 | mg | COX-2 inhibitor |
| 150 | mg | Polyvinylpyrrolidone |
| 2.5 | mg | Poly oxyethylene sorbitan |
| | | monolaurate |
| 10 | mg | Benzoic acid |
| to 5 | mL with sorbitol solution (70%) | |

Suspension dose strengths of between 1 and 50 mg/5 ml can be accommodated by varying the ratio of the first two ingredients.

### EXAMPLE 9

Intravenous infusion

| Amount per 200 mL dose | | Ingredient |
|---|---|---|
| 1 | mg | COX-2 inhibitor |
| 0.2 | mg | Polyethylene oxide 400 |
| 1.8 | mg | Sodium chloride |
| to 200 | mL | Purified water |

### EXAMPLE 10

Combination Tablet Preparation

Tablets containing 25.0, 50.0, and 100.0 mg, respectively, of an inhibitor of activated thrombin activatable fibrinolysis inhibitor, e.g. 3-(6-amino-pyridin-3-yl)-2-mercaptomethyl-propionic acid, and 25 mg of a COX-2 inhibitor, e.g., 3-(3,4-difluorophenyl)-4-(4-(methylsulfonyl)phenyl)-2-(5H)-furanone, are prepared as illustrated below:

| | Amount-mg | | |
|---|---|---|---|
| Inhibitor of activated thrombin | 25.0 | 50.0 | 100.0 |
| activatable fibrinolysis inhibitor | | | |
| COX-2 inhibitor | 25.0 | 25.0 | 25.0 |
| Microcrystalline cellulose | 37.25 | 100.0 | 175.0 |
| Modified food corn starch | 37.25 | 4.25 | 8.5 |

Both active compounds, cellulose, and a portion of the corn starch are mixed and granulated to 10% corn starch paste. The resulting granulation is sieved, dried and blended with the remainder of the corn starch and the magnesium stearate. The resulting granulation is then compressed into tablets containing 25.0, 50.0, and 100.0 mg, respectively, of inhibitor of activated thrombin activatable fibrinolysis inhibitor per tablet, and 25 mg COX-2 inhibitor, per tablet.

### EXAMPLE 11

Patients suffering from pain caused by nephritis, systemic lupus, erythematosus, rheumatoid arthritis, glomerulonephritis, vasculitis and sacoidosis, and the like are treated for this pain by ingesting 50 mg of 3-(6-amino-pyridin-3-yl)-2-mercaptomethyl-propionic acid (e.g. tablet prepared in Example 1), an inhibitor of activated thrombin activatable fibrinolysis inhibitor, once each day.

### EXAMPLE 12

Patients suffering from pain caused by nephritis, systemic lupus, erythematosus, rheumatoid arthritis, glomerulonephritis, vasculitis and sacoidosis, and the like are treated for this pain by ingesting a tablet comprising 50 mg of 3-(6-amino-pyridin-3-yl)-2-mercaptomethyl-propionic acid (e.g. tablet prepared in Example 1), an inhibitor of activated thrombin activatable fibrinolysis inhibitor, in combination with a tablet comprising 25 mg of 3-(3,4-difluorophenyl)-4-(4-(methylsulfonyl)phenyl)-2-(5H)-furanone (e.g. tablet prepared in Example 4), a COX-2 inhibitor, once each day.

### EXAMPLE 13

Patients suffering from pain caused by nephritis, systemic lupus, erythematosus, rheumatoid arthritis, glomerulonephritis, vasculitis and sacoidosis, and the like are treated for this pain by ingesting a tablet comprising 50 mg of 3-(6-amino-pyridin-3-yl)-2-mercaptomethyl-propionic acid (an inhibitor of activated thrombin activatable fibrinolysis inhibitor) and 25 mg of 3-(3,4-difluorophenyl)-4-(4-(methylsulfonyl)phenyl)-2-(5H)-furanone (a COX-2 inhibitor) (e.g. tablet prepared in Example 10), once each day.

While the invention has been described and illustrated with reference to certain particular embodiments thereof, those skilled in the art will appreciate that various changes, modifications and substitutions can be made therein without departing from the spirit and scope of the invention. For example, effective dosages other than the particular dosages as set forth herein above may be applicable as a consequence of variations in the responsiveness of the mammal being treated for any of the indications for the active agents used in the instant invention as indicated above. Likewise, the specific pharmacological responses observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

What is claimed is:

1. A method for treating an inflammatory disease in a patient which comprises treating the patient with a therapeutically effective amount of an inhibitor of activated thrombin activatable fibrinolysis inhibitor.

2. A method of claim 1 wherein the patient is a human.

3. A method for reducing the risk of inflammation in a patient which comprises treating the patient with a therapeutically effective amount of an inhibitor of activated thrombin activatable fibrinolysis inhibitor.

4. A method of claim 3 wherein the patient is a human.

5. A method for treating pain in a patient which comprises treating the patient with a therapeutically effective amount of an inhibitor of activated thrombin activatable fibrinolysis inhibitor.

6. A method of claim 5 wherein the patient is a human.

7. A method for reducing the risk of pain in a patient which comprises treating the patient with a therapeutically effective amount of an inhibitor of activated thrombin activatable fibrinolysis inhibitor.

8. A method of claim 7 wherein the patient is a human.

9. A method of claim 1, wherein the patient has a condition in which provision of an antithrombotic effect is desirable.

10. A method of claim 1, wherein the patient has a condition in which provision of an antithrombotic effect to prevent or reduce the incidence of thrombosis is also desirable.

11. A method of claim 1 wherein the inflammatory disease is selected from the group consisting of nephritis, systemic lupus, erythematosus, rheumatoid arthritis, glomerulonephritis, vasculitis and sacoidosis.

12. A method of claim 1 wherein the inhibitor of activated thrombin activatable fibrinolysis inhibitor is selected from the group consisting of 2-(6-amino-pyridin-3-ylmethyl)-3-butyl-succinic acid, 2-(6-amino-pyridin-3-ylmethyl)-3-phenethyl-succinic acid, 2-(6-amino-pyridin-3-ylmethyl)-3-methyl-succinic acid, 2-(6-amino-5-methyl-pyridin-3-ylmethyl)-3-[(1-benzyloxycarbonylamino-2-methyl-propyl)hydroxy-phosphinoyl]-propionic acid, 2-(6-amino-pyridin-3-ylmethyl)-3-[hydroxy-(3-phenyl-propyl)-phosphinoyl]-propionic acid, 2-(amino-pyridin-3-ylmethyl)—N-hydroxy-succinamic acid, 3-(6-amino-pyridin-3-yl)-2-mercaptomethyl-propionic acid, 2-(2-amino-pyridin-4-ylmethyl)-3-mercapto-propionic acid, 2-(6-amino-pyridin-3-ylmethyl)-2-mercaptomethyl-butyric acid, 3-(6-amino-5-methyl-pyridin-3-yl)-2-mercaptomethyl-2-methyl-propionic acid, 3-(6-amino-5-methyl-pyridin-3-yl)-2-mercaptomethyl-propionic acid, 3-(6-amino-4-methyl-pyridin-3-yl)-2-mercaptomethyl-propionic acid, and 3-(6-amino-pyridin-3-yl)-2-mercaptomethyl-butyric acid or a pharmaceutically acceptable salt thereof.

13. A method for treating an inflammatory disease in a patient which comprises treating the patient with a therapeutically effective amount of a composition comprising an inhibitor of activated thrombin activatable fibrinolysis inhibitor and an NSAID.

14. A method for reducing the risk of inflammation in a patient which comprises treating the patient with a therapeutically effective amount of a composition comprising an inhibitor of activated thrombin activatable fibrinolysis inhibitor and an NSAID.

15. A method for treating pain in a patient which comprises treating the patient with a therapeutically effective amount of a composition comprising an inhibitor of activated thrombin activatable fibrinolysis inhibitor and an NSAID.

16. A method for reducing the risk of pain in a patient which comprises treating the patient with a therapeutically effective amount of a composition comprising an inhibitor of activated thrombin activatable fibrinolysis inhibitor and an NSAID.

17. A method of claim 13 wherein the inflammatory disease is selected from the group consisting of nephritis, systemic lupus, erythematosus, rheumatoid arthritis, glomerulonephritis, and sacoidosis.

18. A method of claim 13 wherein the inhibitor of activated thrombin activatable fibrinolysis inhibitor is selected from the group consisting of 2-(6-amino-pyridin-3-ylmethyl)-3-butyl-succinic acid, 2-(6-amino-pyridin-3-ylmethyl) -3-phenethyl-succinic acid, 2-(6-amino-pyridin-3-ylmethyl)-3-methyl-succinic acid, 2-(6-amino-5-methyl-pyridin-3-ylmethyl)-3-[(1-benzyloxycarbonylamino-2-methyl-propyl)hydroxy-phosphinoyl]-propionic acid, 2-(6-amino-pyridin-3-ylmethyl)-3-[hydroxy-(3-phenyl-propyl)-phosphinoyl]-propionic acid, 2-(amino-pyridin-3-ylmethyl)—N-hydroxy-succinamic acid, 3-(6-amino-pyridin-3-yl)-2-mercaptomethyl-propionic acid, 2-(2-amino-pyridin-4-ylmethyl)-3-mercapto-propionic acid, 2-(6-amino-pyridin-3-ylmethyl)-2-mercaptomethyl-butyric acid, 3-(6-amino-5-methyl-pyridin -3-yl)-2-mercaptomethyl-2-methyl-propionic acid, 3-(6-amino-5-methyl-pyridin-3-yl)-2-mercaptomethyl-propionic acid, 3-(6-amino-4-methyl-pyridin-3-yl)-2-mercaptomethyl-propionic acid, and 3-(6-amino-pyridin-3-yl)-2-mercaptomethyl-butyric acid and the NSAID is a COX-2 inhibitor.

19. A method of claim 18 wherein the COX-2 inhibitor is 3-(3,4-difluorophenyl)-4-(4-(methylsulfonyl)phenyl)-2-(5H)-furanone or a pharmaceutically acceptable salt thereof.

20. A method of claim 18 wherein the COX-2 inhibitor is 3-phenyl-4-(4-(methylsulfonyl)phenyl)-2-(5H)-furanone or a pharmaceutically acceptable salt thereof.

21. A method of claim 18 wherein the COX-2 inhibitor is 5-chloro-3-(4-methanesulfonylphenyl)-6′-methyl-[2,3′]bipyridinyl or a pharmaceutically acceptable salt thereof.

22. A method for treating an inflammatory disease in a patient which comprises treating the patient with a therapeutically effective amount of an inhibitor of activated thrombin activatable fibrinolysis inhibitor in combination with a therapeutically effective amount of a COX-2 inhibitor.
